SEQUENCE LISTING (2 sequences)

SEQ ID NO: 1 - protein, 195 aa, Homo sapiens
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKND

SEQ ID NO: 2 - DNA, 588 bp, Homo sapiens
atggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattaa

(19) United States
(12) Patent Application Publication
(10) Pub. No.: US 2012/0015363 A1
(43) Pub. Date: Jan. 19, 2012

(54) Progesterone receptor membrane component 1(PGRMC1) as an indicator of human fertility

(75) Inventor: John J. Peluso, Avon, CT (US)

(73) Assignee: University of Connecticut, Farmington, CT (US)

(21) Appl. No.: 13/135,747

(22) Filed: Jul. 14, 2011

Related U.S. Application Data
(60) Provisional application No. 61/364,708, filed on Jul. 15, 2010.

(51) Int. Cl. C12Q 1/68; G01N 33/58; G01N 33/566

(52) U.S. Cl. 435/6.12; 435/7.21; 435/40.5

(57) ABSTRACT

Methods of evaluating fertility of a human based on determination of a PGRMC1 characteristic that correlates with human fertility. In one embodiment, a sample is obtained from a human, a PGRMC1 characteristic is determined and compared to a baseline PGRMC1 characteristic, wherein a variation between the determined PGRMC1 characteristic and the baseline characteristic indicates a level of fertility of the human. A PGRMC1 characteristic can be one or more of PGRMC1 expression, transcription, translation, amino acid sequence, nucleic acid sequence, post-translational modification, cell localization or tissue localization. A sample can be a cell sample, a tissue sample, a blood sample, a lymphocyte sample, an oocyte sample, or a sperm sample. A human can be male or female. In one embodiment, a variation of PGRMC1 nucleic acid sequence indicates reduced fertility of the human.

### RELATED APPLICATIONS

The present application claims the benefit of U.S. Provisional Patent Application Ser. No. 61/364,708, filed Jul. 15, 2010, which is herein incorporated by reference in its entirety.

### STATEMENT OF GOVERNMENT SUPPORT

This invention was made with government funding under the NIH grant entitled “Progesterone regulation of human luteal cell viability” (NIH grant number R03HD050298) and NIH grant entitled “PAIRBP1 & PGRMC1 act as a membrane receptor complex to mediate P4's ovarian action” (NIH grant number R01 HD 052740). The government has certain rights in the invention.

### FIELD

Embodiments of the present invention relate to methods of evaluating, assessing or predicting human fertility based on analysis of progesterone receptor membrane component 1 (PGRMC1).

### BACKGROUND

Fertility problems affect 15% of human couples of childbearing age. Assisted reproductive technologies, such as in vitro fertilization, are available, but they are often costly and time-consuming procedures, with a success rate that is difficult to predict. For example, in vitro fertilization consists of two phases. First, women are treated with gonadotropins to induce follicular growth and oocyte maturation. This is followed by the retrieval of oocytes that are subsequently fertilized and developed in vitro prior to being transferred into the patient's uterus. For an in vitro fertilization protocol to be successful, at a minimum, gonadotropin treatment must lead to the production of viable, fertilizable oocytes. However, many women fail to respond adequately to the gonadotropin treatment and do not produce oocytes that are fertilizable and/or have the potential for normal embryonic development. For the women who do not generate such oocytes, fertility treatments are an expensive, painful, emotionally frustrating and, ultimately, futile experience.

At present few tests are available to assess the ability of a female to generate oocytes that can be fertilized and develop into normal offspring, or of a male's ability to generate sperm capable of fertilizing an oocyte that can result in normal offspring. Other than becoming pregnant through sexual interaction with a male or artificial insemination with sperm, one of the few ways to assess fertility of a female is to undergo fertility treatments, such as in vitro fertilization protocols, in which the fertilizability of the oocyte is determined by exposure to sperm in culture. No genetic tests are available at present to assess fertility in human patients.

Currently available methods of fertility assessment are based on monitoring known clinical markers, such as changes in estradiol levels. However, these tests do not directly monitor a substance produced by oocytes. Moreover, they have limited predictive value. For example, during in vitro fertilization procedures the patients are usually required to undergo a “test” procedure. If the “test” in vitro fertilization procedure does not yield fertilizable oocytes, then a retrospective analysis of the “test” in vitro fertilization cycle, including the clinical markers, is undertaken. This analysis is then applied to modify the procedure for use in a second in vitro fertilization protocol. This is essentially a “hit or miss” approach that imposes emotional and financial hardships on the patients.

One of the major difficulties in developing tests for assessing probability of a successful outcome of a fertility treatment, including an assisted reproductive technique, for example, in vitro fertilization, lies in complex, varied and ill defined causes of infertility. Of the different types of infertility, as classified in the Society for Assisted Reproductive Technology (SART) database, diminished ovarian reserve (reduced number of ovarian follicles) is the only type of infertility for which tests are beginning to be developed. These tests involve monitoring serum levels of follicle-stimulating hormone (FSH), inhibin B and anti-Müllerian hormone. If these serum measurements suggest a reduced ovarian follicle population, then this is interpreted as an indicator that the patient is not likely to respond well to the “standard” gonadotropin treatment. The results of the FSH/inhibin B/anti-Müllerian hormone tests can then be used to justify either encouraging the patient not to undergo the fertility treatments, such as in vitro fertilization protocol, or changing the gonadotropin protocol.

Unfortunately, infertility patients with diminished ovarian reserve are known to only account for a small percentage (≈1%) of the total number of patients. According to the available data published by the Center for Advanced Reproductive Services at the University of Connecticut Health Center (Farmington, Conn.), of the remaining patients seen at the infertility clinic at the University of Connecticut Health Center in 2007, only 56% had an identified cause of their infertility, which included male factor (12%), tubal factor (11%), endometriosis (12%), uterine factor (1%), other known factors (11%), and ovulatory dysfunction (8%). The remaining 44% of the 1100 cycles were from patients without an infertility diagnosis and were classified as unexplained infertility. Without identifying the etiology of their infertility, it is difficult to develop a test to predict the outcome of an in vitro fertilization protocol.

What is needed is a test to evaluate the probability of whether an individual female will produce viable oocytes capable of being fertilized and developing into viable embryos (“functional oocytes”). What is also needed is a test to evaluate a probability of whether an individual male's ability to produce sperm cells capable of fertilizing oocytes so that they can develop into viable embryos (“functional sperm cells”). What is also needed is a test that would predict the probability of a successful outcome of a fertility treatment of an individual patient or a couple, based on the ability to produce functional oocytes, functional sperm cells, or both. What is also needed is a test that would allow customization of fertility treatments for individual patients or couples based on their ability to produce functional oocytes, functional sperm, or both. In the in vitro fertilization context, a test is needed that would predict a female patient's ability to respond to gonadotropin treatment and produce functional oocytes. For infertile women, including but not limited to those who are not diagnosed with diminished ovarian reserve, a test is needed that will evaluate outcomes of fertility treatments, such as in vitro fertilization outcomes. A test is also needed that would be relatively non-invasive, such as, for example, the testing of blood samples.

### SUMMARY

Disclosed herein are embodiments of methods of assessing and/or evaluating fertility of humans. For example, disclosed herein are methods of assessing or evaluating the ability of humans to produce functional gametes (eggs or sperm) by analyzing characteristics of progesterone receptor membrane component-1 (PGRMC1). Some examples of PGRMC1 characteristics used in embodiments of the methods disclosed herein are levels of PGRMC1 expression, including transcription and translation, protein or nucleic acid structure or sequence, post-translational modifications, localization in cells and tissues. In some embodiments, methods disclosed herein evaluate, measure, assess or determine variation of PGRMC1 characteristics. In the context of the methods disclosed herein, PGRMC1 variation encompasses, without limitation, variation in PGRMC1 expression, including transcription and translation, variation of protein or nucleic acid structure, variation of post-translational modifications, or variation in localization in cells and tissues. Generally, variation is understood to imply the differences in certain PGRMC1 characteristics, such as those listed above, identified in a particular individual with respect to the baseline, typical, or average characteristics, or temporal variation in PGRMC1 characteristics in an individual human, or in a cell or tissue, including a cell or a tissue sample obtained from a human.

In one embodiment, analysis of PGRMC1 characteristics and their variation is used to predict the capacity of a female to produce functional oocytes, or oocytes that are able to be fertilized and ultimately develop into a healthy offspring. In another embodiment, analysis of PGRMC1 characteristics and/or their variation is used to predict the capacity of a male to produce functional sperm cells, or sperm cells that are able to fertilize oocytes, which are then able to develop into healthy offspring. In yet another embodiment, a method to improve the efficiency of fertility treatments or assisted reproduction techniques, such as in vitro fertilization protocols, is provided by analyzing PGRMC1 characteristics and/or their variation in a patient. In one more embodiment, a method of predicting outcome of fertility treatments or assisted reproduction techniques, such as in vitro fertilization protocols, is provided by analyzing PGRMC1 characteristics and/or their variation. In one more embodiments, a method of selecting functional gametes is provided by analyzing PGRMC1 characteristics and/or their variation.

Also provided are methods for diagnosing infertility in a human that include analyzing PGRMC1 characteristics and/or their variation in the human. In yet another embodiment, a method of treating infertility is provided by improving the effectiveness of an infertility treatment or an assisted reproduction technique in a patient that includes analyzing PGRMC1 characteristics and/or their variation in the patient and modifying the treatment or the technique in accordance with the results of PGRMC1 analysis. These and other features and advantages of the present methods will become apparent after a review of the following detailed description of the disclosed embodiments and the appended claims.

### BRIEF DESCRIPTION OF THE FIGURES

FIG. 1 is a reproduction of an image of a Western blot showing PGRMC1 expression in bovine ovarian cortex and bovine oocytes. A Western blot analysis conducted in the absence of the PGRMC1 antibody, is shown as a negative control (−).

FIG. 2 is a reproduction of epi-fluorescent images of cells showing localization of PGRMC1 in bovine and mouse germinal vesicle-stage oocytes. PGRMC1 is shown in red. PGRMC1 was detected throughout the oocyte, but is highly concentrated within the germinal vesicle (arrows) of both bovine and mouse oocytes. Negative controls were conducted on both bovine and mouse oocytes by omitting the primary antibody, which did not reveal any staining. Mag bar is 20μ.

FIG. 3 is a reproduction of confocal images of cells showing changes in the localization of PGRMC1 during bovine oocyte maturation. A negative control, which was conducted in the absence of the PGRMC1 antibody, is shown in the upper left panel. The DNA was stained with DAPI and is shown in blue, and PGRMC1 is shown in red. Oocytes were collected at the germinal vesicle stage (GV), after the breakdown of the GV (i.e. GVBD), prometaphase 1 (Pro-MI), metaphase I (Meta I), anaphase I (Ana I), telophase I (Telo I) and metaphase II (Meta II). The images of metaphase I, anaphase I and metaphase II are shown in a polar view (i.e. looking down onto the metaphase chromosomes). The images of the anaphase I and telophase I oocytes are in the lateral view (i.e. looking at the side of the anaphase I and telophase I chromosomes).

FIG. 4 is a reproduction of confocal images of cells showing colocalization of PGRMC1 and Aurora B on metaphase II chromosomes in bovine oocytes. The chromosomes are shown in blue, PGRMC1 in red and Aurora B in green. A merged image of PGRMC1 and Aurora B staining is shown in the lower right panel. The areas that appear orange-yellow are areas where PGRMC1 and Aurora B colocalize. Mag bar=10μ.

FIG. 5 is a reproduction of epi-fluorescent images of cells showing PGRMC1 localization in a bovine zygote. PGRMC1 (red) is only detected within nucleolar-like structures of the female and male pronuclei.

FIG. 6 is a reproduction of epi-fluorescent images of the cells showing localization of PGRMC1 in a bovine blastocyst. PGRMC1 is shown in red (A) and nuclei are shown in blue (B). A merged image is shown in C.

FIG. 7 is a bar graph representing the data on an effect of PGRMC1 antibody injection on bovine oocyte maturation. On the x-axis, the following abbreviations are used: GVBD: germinal vesicle breakdown: proMI: prometaphase I; MI: metaphase I; A/T: anaphase/telophase; MII: metaphase II. * indicates a difference between control and PGRMC1 antibody injection (p<0.05).

FIG. 8 is a reproduction of epi-fluorescent images of cells showing the effect of PGRMC1 antibody injection on the alignment of chromosomes along the metaphase I during bovine oocyte maturation in vitro. Oocytes were stained with propididum iodide (red) to reveal the chromosomes. Panel A shows chromosomes precisely arranged in a metaphase I plate, which is typical of IgG injected oocytes. Panel B shows a bovine oocyte 24 h after being injected with PGRMC1 antibody, with the chromosomes not aligned and the metaphase plate disorganized.

FIG. 9 is a reproduction of a fluorescent image of granulosa/luteal cells in culture showing protein expression as a result an infection with adenovirus-PGRMC1-GFP construct a MOI of 1×10⁻⁷, resulting in nearly 100% of the cells expressing PGRMC1-GFP fusion.

FIG. 10 is a bar graph schematically representing variation in the fertilization rate of women with the diagnosis of either unexplained or tubal infertility.

### DETAILED DESCRIPTION

Disclosed herein are embodiments of methods of assessing or evaluating fertility, including the ability to produce functional gametes, of humans that include analyzing characteristics of PGRMC1. In some embodiments, methods of assessing or evaluating fertility include determination and analysis of variation of one or more PGRMC1 characteristic. It is understood that analyzing PGRMC1 characteristics and/or their variation allows assessment of an oocyte's capacity to develop within an ovarian follicle and then undergo maturation, fertilization and normal embryological development, and of a sperm cell's capacity to develop and ultimately fertilize an oocyte, which is then able to undergo normal embryological development. Gametes having such capacities are referred as “functional gametes.” It is understood that the ability of a human to produce functional gametes correlates with fertility and with the success rate of assisted reproductive techniques and fertility treatments, including the outcome of in vitro fertilization.

It is discovered and described herein that PGRMC1 plays an essential role in regulating oocyte development and function. In particular, it is discovered that PGRMC1 influences an oocyte's capacity to undergo maturation, fertilization and normal embryological development. In mammalian ovaries, oocytes are arrested in prophase of the first meiotic division until they reach their full size and enter the preovulatory meiotic maturation process. Nuclear meiotic maturation of oocytes includes condensation of chromosomes, germinal vesicle breakdown (GVBD), progression through prometaphase I and metaphase I, the transition throughout anaphase I and telophase I and an arrest at metaphase II. This process is coordinated by several kinases and initiates when the luteinizing hormone surge stimulates the resumption of meiosis in one or more oocytes depending on the species. In mammalian oocytes, fully-grown oocytes are capable of resuming meiosis in vitro, and then of being fertilized and developing to the blastocyst stage. During oocyte meiotic division, the meiotic spindle asymmetrically segregates homologous pairs into the secondary oocyte and the polar body, then the egg arrests to metaphase II until fertilization occurs.

Defects in spindle formation can generate chromosome instability and aneuploidy, a condition known to be the major cause of miscarriages and birth defects. For example, aneuploidy is observed in a large percentage (as high as 40%) of embryos derived from patients undergoing infertility treatment. To guarantee the correct function of the spindle, the activity and localization of spindle-associated proteins has to be strictly regulated in time and space. The studies described herein are the first to show that PGRMC1 is 1) expressed in mammalian oocytes and 2) associated with the meiotic spindle. Moreover, it is discovered and described herein that PGRMC1 's localization dramatically changes during oocyte maturation, fertilization and early preimplantation development.

For example, PGRMC1 localizes to the centromere of chromosomes in metaphase I and metaphase II oocytes. Localization to centromeres is significant because centromeres are involved in the attachment of microtubules, which function to separate the chromosomes during the metaphase to telophase transition. The temporal changes in the expression of PGRMC1 during oocyte maturation, fertilization and early preimplantation development indicate an important role for PGRMC1. For example, dramatic changes in localization indicate that PGRMC1 is involved in regulating the separation of chromosomes during oocyte maturation. PGRMC1 is also found in sperm cells, as discussed, for example, in Lösel et al., “Classic and Non-classic Progesterone Receptors are Both Expressed in Human Spermatozoa,” Horm. Metab. Res. 37:10-14, 2005; Lösel et al. “Porcine Spermatozoa Contain More than One Membrane Progesterone Receptor,” Int. Journ. Of Biochemistry and Cell Biology, 36:1532-1541, 2004. Accordingly, PGRMC1 protein and nucleic acids encoding PGRMC1 are useful as markers of a capacity of a human to correctly undergo various processes involved in production and functioning of gametes, including the processes discussed in above, as well as of the functioning of zygotes and embryonic development.

Embodiments of methods of analyzing or testing of PGRMC1 characteristics and/or their variation are described herein. Embodiments of the methods described herein are useful for assessing functioning of the biological processes discussed above, some or all of which are understood to be important for fertility of a human. In other words, certain embodiments of the methods described herein allow assessing the capacity of oocytes and sperm to undergo various processes affecting fertility, such as, but not limited to, meiosis, fertilization and cell differentiation. Thus, certain embodiments of the described methods allow for assessment of the ability of the oocytes and sperm to undergo fertilization, implantation, as well as to generate normal embryos.

### PGRMC1 Sequences and Mutations

Polypeptide and nucleic acid sequences for PGRMC1 are known in the art and can be obtained from publicly available sources. For example, such as, polypeptide and nucleic acid sequence databases are available through the National Center for Biotechnology Information (NCBI).

An example of a polypeptide sequence for human PGRMC1 is GenBank Accession No. CAG33274:

(SEQ ID NO: 1)
MAAEDVVATGADPSDLESGGLLHEIFTSPLNLLLLGLCIFLLYKIVRGDQ
PAASGDSDDDEPPPLPRLKRRDFTPAELRRFDGVQDPRILMAINGKVFDV
TKGRKFYGPEGPYGVFAGRDASRGLATFCLDKEALKDEYDDLSDLTAAQQ
ETLSDWESQFTFKYHHVGKLLKEGEEPTVYSDEEEPKDESARKND

An example of a nucleotide sequence for human PGRMC1 is GenBank Accession No. CR456993 (stop codon “taa” is indicated):

(SEQ ID NO: 2)
atggctgccg aggatgtggt ggcgactggc gccgacccaa
gcgatctgga gagcggcgggctgctgcatg agattttcac
gtcgccgctc aacctgctgc tgcttggcct
ctgcatcttcctgctctaca agatcgtgcg cggggaccag
ccggcggcca gcggcgacag cgacgacgacgagccgcccc
ctctgccccg cctcaagcgg cgcgacttca cccccgccga
gctgcggcgcttcgacggcg tccaggaccc gcgcatactc
atggccatca acggcaaggt gttcgatgtgaccaaaggcc
gcaaattcta cgggcccgag gggccgtatg gggtctttgc
tggaagagatgcatccaggg gccttgccac attttgcctg
gataaggaag cactgaagga tgagtacgatgacctttctg
acctcactgc tgcccagcag gagactctga gtgactggga
gtctcagttcactttcaagt atcatcacgt gggcaaactg
ctgaaggagg gggaggagcc cactgtgtactcagatgagg
aagaaccaaa agatgagagt gcccggaaaa atgattaa

Naturally occurring mutations in PGRMC1 are known to exist. Such mutations have been identified and described in human females with known fertility problems. For example, the studies on alteration in expression, structure and function of PGRMC1 in females with premature ovarian failure are summarized in Mansouri, M. R., et al., “Alterations in the expression, structure and function of progesterone receptor membrane component-1 (PGRMC1) in premature ovarian failure,” Hum. Mol. Genet, 17(23):3776-83, 1998. Premature ovarian failure (POF) is a condition characterized by hypergonadotropic hypogonadism and amenorrhea in human females before the age of 40. In a scientific study, a mother and daughter with POF were identified both of whom carried an X; autosome translocation [t(X;11)(q24;q13)] and had reduced expression levels of PGRMC1, as determined based on RNA transcript and protein levels. A human female with POF was also identified carrying a missense mutation (H165R) located in the cytochrome b5 domain of PGRMC1, which was shown to be associated with abolition of the binding of PGRMC1 to cytochrome P450 7A1 (CYP7A1). PGRMC1 is known to be positive regulators of several cytochrome P450 (CYP)-catalyzed reactions. The H165R mutation is also known to attenuate PGRMC1 's ability to mediate the anti-apoptotic action of progesterone in ovarian cells. According to some embodiments of the methods described herein, at least some genetic alterations in PGRMC1 adversely affect the ability of the oocyte to mature, fertilize and undergo early preimplantation development.

In addition to H165R, another missense mutation at amino acid 120 has been detected, which results in a complete loss of PGRMC1 's actions, as discussed in Peluso et al., “Progesterone receptor membrane component-1 (PGRMC1) is the mediator of progesterone's anti-apoptotic action in spontaneously immortalized granulosa cells as revealed by PGRMC1 small interfering ribonucleic acid treatment and functional analysis of PGRMC1 mutations,” Endocrinology 149:534-543, 2008 (“Peluso I”). Accordingly, certain embodiments of the present method provide methods of assessing fertility that include testing of PGRMC1 in order to determine the presence of mutations in the PGRMC1-encoding nucleic acid sequences.

### PGRMC1 Characteristics

Embodiments of the methods provided herein evaluate one or more of the following PGRMC1 characteristics: structure and function of cells or tissues containing PGRMC1 protein and PGRMC1-encoding nucleic acids; expression of PGRMC1 protein; levels of PGRMC1 protein; location of expression of PGRMC1 protein; transcription of PGRMC1 mRNA; levels of PGRMC1 mRNA; location of PGRMC1 mRNA; structure and function of PGRMC1 protein; structure and function of PGRMC1-encoding nucleic acids, including DNA and RNA; interactions of PGRMC1 protein and PGRMC1-encoding nucleic acids in with other proteins, nucleic acids or other molecules; structure and function of chromatin and chromosomes containing PGRMC1-encoding nucleic acids.

### PGRMC1 Analysis or Testing

Evaluation of the foregoing characteristics according to embodiments of the methods disclosed herein can be performed in whole organisms, tissues, cells, samples obtained from organisms, tissues or cells, in various in situ, in vivo and in vitro systems, and in computational modeling systems (also referred to as in silico). All of the foregoing is collectively referred to as “PGRMC1 analysis” or “PGRMC1 testing.” Embodiments of the present methods utilize PGRMC1 testing in order to determine if variation in any of the foregoing PGRMC1 characteristics exists in an individual human, or in a cell or a tissue, including a cell or a tissue sample obtained from a human, as compared to average or normal characteristics existing in a human population. Other embodiments of the present methods utilize PGRMC1 testing in order to determine temporal or spatial variation of PGRMC1 in an individual human, or in a cell or a tissue, including a cell or a tissue sample obtained from a human.

In certain embodiments of the methods described herein, analysis or testing of PGRMC1 characteristics and/or their variation is conducted in a sample obtained from a human. A sample is a cell or tissue sample containing PGRMC1, PGRMC1-encoding nucleic acids, or both. One advantage of certain embodiments of the methods discussed herein is that PGRMC1 protein is generally expressed in oocytes and sperm, which permits assessment of fertility of both males and females. Another advantage of certain embodiments of the methods discussed herein is that PGRMC1 is present within blood cells, for example, lymphocytes, which allows for convenient and relatively non-invasive testing of a blood sample. However, testing of other cells and tissues where PGRMC1 is present, such as the sperm cells and the oocytes, can also be conducted. Testing of PGRMC1-encoding nucleic sequences can be conducted on any cells and tissues where such sequences are present. Yet another advantage of certain embodiments of the methods discussed herein is that they allow assessing the capacity of oocytes and sperm to undergo various processes affecting fertility, such as, but not limited to, meiosis, fertilization and cell differentiation. Thus, certain embodiments of the discussed methods allow identification of the specific biological processes that are diminished or defective in human infertility patients.

### Diagnostic Methods

Diagnostic methods used in the embodiments of the method described herein include, but are not limited to, the following techniques: competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunocytochemistry, immunohistochemistry, PCR, quantitative PCR, real-time PCR, quantitative real-time PCR, in situ PCR of tissue or cell samples, and the like. The skilled artisan will understand that any antibody-based, nucleic acid-based, mass spectroscopy-based, FRET-based, or similar technique for detecting PGRMC1 levels can be used in the embodiments of the methods described herein.

It is appreciated, as exemplified by certain findings discussed herein, that PGRMC1 plays a role in one or more of oocyte and sperm production and function, gamete function, or embryonic development. In one embodiment of the present method, PGRMC1 testing is conducted in humans in order to assess fertility or in connection with fertility treatments.

### Fertility

Unless otherwise qualified, the term “fertility” refers generally to the natural capability of giving life. In live organisms, fertility is influenced by multiple biological processes, including, without limitation, gamete production, fertilization, embryonic development, or an ability to carry a pregnancy to term. Fertility is also influenced by various other factors, such as, but not limited to, nutrition, sexual behavior, culture, instinct, endocrinology, timing, living conditions or emotions. Reproductive hormones participate in fertility regulation by various mechanisms. PGRMC1 is involved in such mechanisms. Mammals have hormonal cycles which determine when a female can achieve pregnancy or when a male is most virile. In humans, for example, the female cycle is approximately twenty-eight days long, but the male cycle is variable.

Unless otherwise qualified, the term fertility encompasses definitions and uses of this term in medical and biological areas. It is appreciated that the term fecundity can also be used to refer to fertility, for example, in the area or demographics, where “fecundity” is commonly defined as the potential for reproduction. Fecundity is included within the scope of the term “fertility,” as used herein. Fertility can be reduced or impaired by various factors generally discussed above as well as by other factors or processes.

Unless otherwise qualified, infertility refers to deficient, lowered, reduced or impaired fertility, as well as to improbability to conceive. The term “infertility” can refer to the biologically reduced ability of a human to contribute to conception, including reduced capacity for production of viable and functioning gametes, zygotes or embryos, as well as to the reduced capacity to carry pregnancy to full term. “Infertility” encompasses various definitions of infertility as used in the medical area. For example, in the medical area, a human couple can be designated as “infertile” in the following situations; if they have not conceived after twelve months of contraceptive-free intercourse and the female is under the age of 34; if the couple has not conceived after 6 months of contraceptive-free intercourse; and the female is over the age of 35, or if the female is incapable of carrying a pregnancy to term. Terms such as, but not limited to, “subfertility,” “reduced fertility” or “impaired fertility,” are also included within the scope of the term “infertility.” For example, in a medical area, a couple that has tried unsuccessfully to have a child for a year or more can be referred to as “subfertile,” meaning less fertile than a typical couple. Infertility includes both primary and secondary infertility. In the medical area, couples that have never been able to conceive can be referred to as “having primary infertility,” while the term “secondary infertility” is often used to refer to difficulty conceiving after already having conceived.

The term “fertility treatment” is used to denote all methods that involve manipulation of human fertility to achieve a desired reproductive result. A human subjected to a fertility treatment can have normal, increased or reduced fertility. “Fertility treatment” as used herein can be used to manipulate fertility to achieve, for example, a desired genetic or other outcome, such as gender or timing of reproduction, or reduction in a risk of infection. “Fertility treatment” encompasses “assisted reproductive technology,” which is used as a general term referring to methods used to achieve fertilization and/or pregnancy by artificial or partially artificial means. In vitro fertilization (IVF) is the term generally used to refer to a process by which egg cells are fertilized by sperm outside the womb, in vitro. IVF process usually includes hormonally controlling the ovulatory process, removing ova (eggs) from the woman's ovaries and permitting sperm to fertilize them in a fluid medium.

Some exemplary embodiments of methods disclosed herein are discussed below. One embodiment is a method of evaluating fertility of a human, comprising obtaining a sample from the human, determining a PGRMC1 characteristic in the sample, and comparing the determined PGRMC1 characteristic to a baseline PGRMC1 characteristic, wherein a variation between the determined PGRMC1 characteristic and the baseline characteristic indicates a level of fertility of the human. One more exemplary embodiment is a method of evaluating capacity of a human to produce functional gametes, comprising determining a characteristic of PGRMC1 of the human, wherein the characteristic indicates the capacity of the human to produce the functional gametes. One more embodiment is a method of evaluating an outcome of a fertility treatment in a human patient comprising determining a characteristic of PGRMC1 of the human patient, wherein the characteristic indicates a capacity of the human patient to produce functional gametes. Embodiments of the disclosed methods encompass variations where a human is a male or a female, and the gametes are eggs or sperm. According to embodiments of the present methods, a PGRMC1 characteristic includes, but is not limited to one or more of the following: PGRMC1 expression, transcription, translation, amino acid sequence, nucleic acid sequence, post-translational modification, cell localization or tissue localization. In one example, PGRMC1 characteristic is a level of PGRMC1 expression, and an either an elevated or a reduced level of PGRMC1 expression in a human as compared to a reference population indicates reduced fertility of the human as compared to the reference population. In another embodiment, the PGRMC1 characteristic to be analyzed or tested is a nucleic acid or a protein sequence and a variation of the nucleic acid or the protein sequence indicates reduced fertility of the human. One example of such variation is H165R mutation or D120G mutation in the human.

### EXAMPLES

Embodiments of the present methods are further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention.

Experimental procedures described in this and other examples are known to those of ordinary skill in the art and are described, for example, in the articles in Peluso I, Peluso et al. “Regulation of ovarian cancer cell viability and sensitivity to cisplatin by progesterone receptor membrane component-1,” J. Clin. Endocrinol. Metab. 93:1592-1599, 2008 (“Peluso II”); Peluso et al. “Progesterone activates a progesterone receptor membrane component 1-dependent mechanism that promotes human granulosa/luteal cell survival but not progesterone secretion,” J. Clin. Endocrinol. Metab., 94:2644-2649, 2009 (“Peluso III”); Peluso et al. “Progesterone Membrane Receptor Component I Expression in the Immature Rat Ovary and Its Role in Mediating Progesterone's Antiapoptotic Action,” Endocrinology, 147:3133-3140 (“Peluso IV”), as well as in other sources referenced below.

### Example 1

### Localization of PGRMC1 in Bovine and Mouse Oocytes

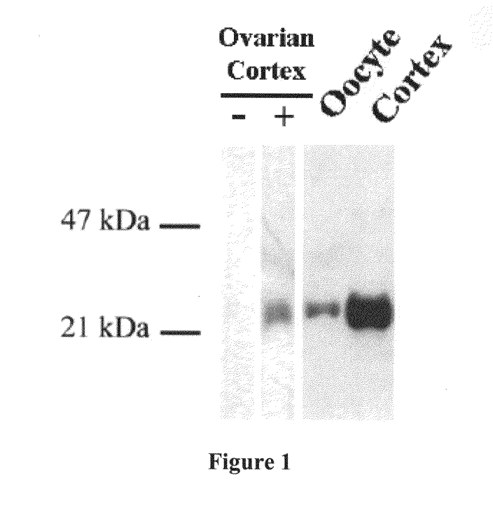

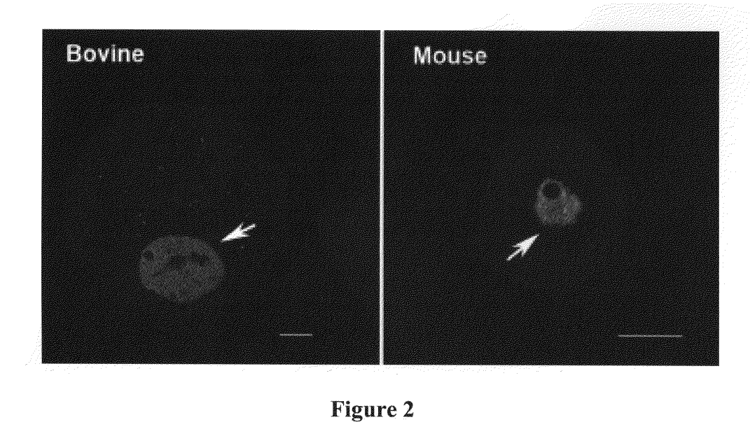

A Western blot analysis with an anti-PGRMC1 antibody (Prestige Antibodies Cat. No. HPA002877, Sigma Chemical Co. St. Louis, Mo.) demonstrated that PGRMC1 was specifically detected as an approximately 26 kDa band in bovine oocytes, as shown in FIG. 1, thus establishing that the antibody specifically detected bovine PGRMC1. The antibody was then used in antibody cell-imaging studies to localize PGRMC1 in oocytes, zygotes and blastocysts. In particular, cell imaging determined localization of PGRMC1 in germinal vesicle stage bovine and mouse oocytes. As seen in FIG. 2, PGRMC1 (shown in red) was highly concentrated within the germinal vesicle of both bovine and mouse oocytes. Similar studies were conducted with other PGRMC1 antibodies using other types of cells and provided similar findings.

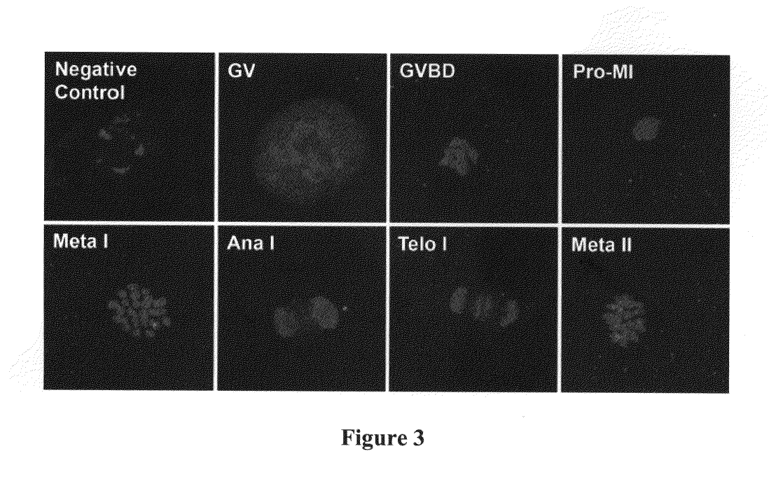

Cell-imaging analysis of localization of PGRMC1 during bovine oocyte maturation, as shown in FIG. 3, revealed a relationship between PGRMC1 (shown in red) and chromatin (shown in blue). Bovine oocytes were collected at the germinal vesicle stage, after the breakdown of the GV, prometaphase 1, metaphase I, anaphase I, telophase I and metaphase II. In the germinal vesicle stage, PGRMC1 does not interact with the chromatin. At prometaphase 1, PGRMC1 started to interact with the chromatin. At metaphase I, it was detected throughout each chromosome, as indicated by the pink-purple staining. After the chromosomes separate in anaphase I and telophase I, PGRMC1 dissociated from the chromosomes and concentrated between them. Finally, in metaphase II, PGRMC1 re-associated with the chromosomes at focal points near the apparent centromeric region of each chromosome. These sequential changes in the localization of PGRMC1 indicated that PGRMC1 plays a role in chromosome separation. The localization of PGRMC1 to focal points near the apparent centromeric region of each chromosome indicated that PGRMC1 colocalizes with the centromere.

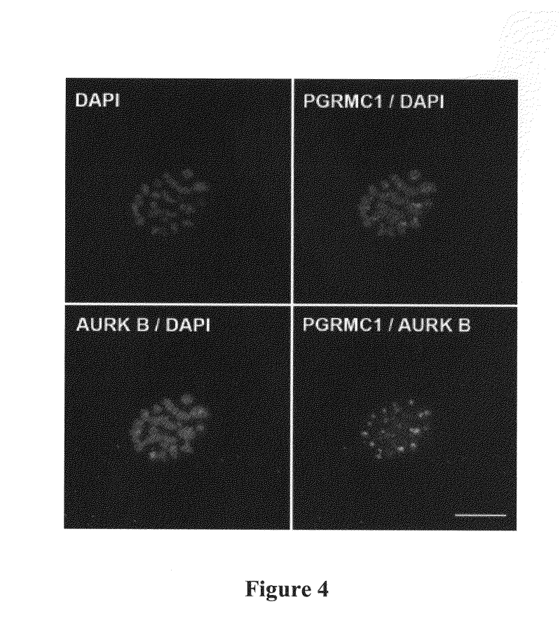

Cell-imaging studies of co-localization of PGRMC1 with Aurora B, a kinase and a well-characterized component of the chromosomal passenger complex that associates with the centromeres, demonstrated that PGRMC1 localizes to Aurora B, as shown in FIG. 4. A centromere is known to be the site at which the kinetochore forms to allow the attachment of spindle fibers for the separation of the chromosomes. The experimental results discussed in this example indicated that that PGRMC1 plays an important role in regulating oocyte maturation.

### Example 2

### Localization of PGRMC1 in a Bovine Zygote

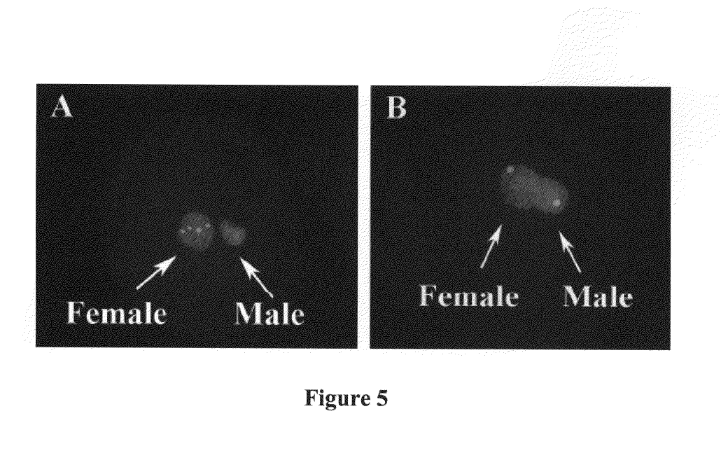

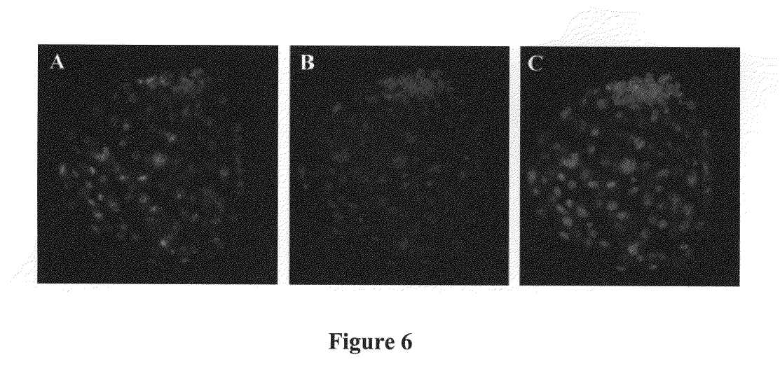

Cell-imaging studies images of PGRMC1 localization in a bovine zygote were performed. As shown in FIG. 5, after in vitro fertilization, PGRMC1 localized almost exclusively to nucleolar-like structures within the female and male pronuclei. As shown in FIG. 6, in blastocysts, PGRMC1 is expressed in virtually all of the cells. The stage-dependent changes in PGRMC1 expression and localization described in this example indicated that PGRMC1 plays important roles in fertilization and early embryonic development as well as in oocyte maturation.

### Example 3

### Role of PGRMC1 in Oocyte Maturation

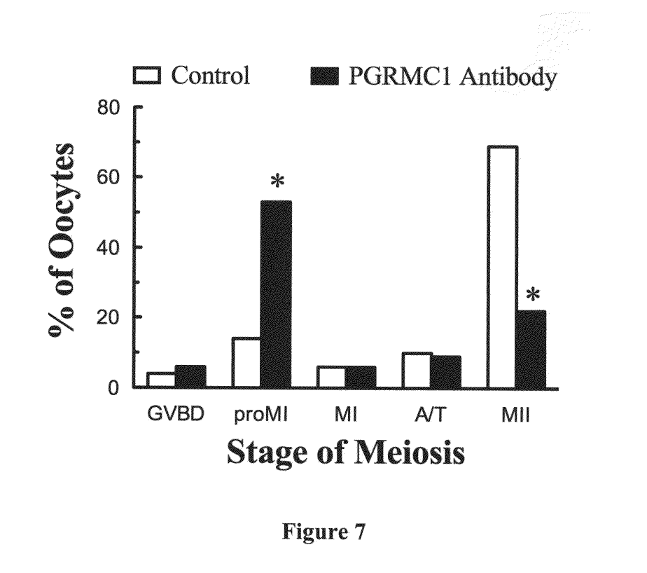

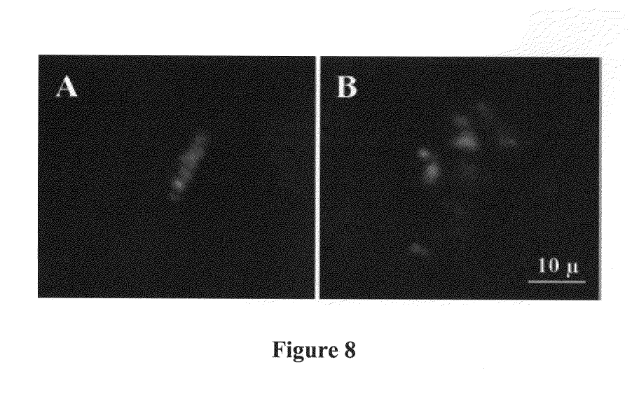

Germinal vesicle (GV) stage bovine oocytes within the cumulus cell mass were injected with an antibody to PGRMC1 (0.3 μM; Sigma Chemical Co, St. Louis, Mo.). Control cells were injected with 0.3 μM IgG. The oocyte-cumulus cell complexes were incubated for 24 hours, then the cumulus cells were removed and the oocytes were assessed for the stage of meiosis. As shown in FIG. 7, 70% of the control oocytes matured to the metaphase II stage. However, only 22% of the PGRMC1 antibody-injected oocytes reached the metaphase II stage (p<0.05). Most of these oocytes were arrested in prometaphase I stage. Some of the PGRMC1 antibody-injected oocytes progressed to metaphase I or II, but cell-imaging studies showed that the metaphase plates of these cells were disorganized, and the chromosomes appeared scattered, as shown in FIG. 8. The experimental results described in this example confirm that PGRMC1 plays a role in oocyte maturation.

### Example 4

### Expression of PGRMC1 During Oocyte Maturation, Fertilization and Development In Vitro in Mouse Oocytes

Adult female mice are injected i.p. with 10 IU of equine chorionic gonadotropin (eCG). Forty-hours later, the germinal vesicle staged oocytes are isolated by puncturing the large antral follicles with a 26 gauge needle. These oocytes are cultured according to a procedure described, for example, in Cao et al., “Cell cycle-dependent localization and possible roles of the small GTPase Ran in mouse oocyte maturation, fertilization and early cleavage,” Reproduction, 130(4):431-440, 2005. Groups of oocytes are removed from culture at two hour intervals over a 12 hour culture period. Once removed from culture, the cumulus cells are removed by incubation with 300 IU/ml of hyaluronidase. The denuded oocytes are then fixed and stained for PGRMC1 and DNA. Cell-imaging studies monitoring changes are conducted to evaluate colocalization of PGRMC1 at certain stages of oocyte maturation with one or more cell structures known to be functionally significant in this process.

### Example 5

### Expression of PGRMC1 during Oocyte Maturation, Fertilization and Development In Vivo in Mouse Oocytes

To monitor changes in PGRMC1 expression during oocyte maturation, adult female CD-1 mice are injected intraperitoneally (i.p.) with 10 IU of equine chorionic gonadotropin (eCG) and 48 hours later with 10 IU of human chorionic gonadotropin (hCG) i.p. At 0, 2, 4, 6, 8, 10 and 12 hours after hCG injection, mice are exposed to carbon dioxide and then cervically dislocated. The oocytes are isolated by puncturing the large antral follicles with a 26 g needle. In addition, ovulated (metaphase II) oocytes enclosed within the cumulus mass are released from the ampulla of the oviduct 24 h after hCG injection. The cumulus-enclosed oocytes are denuded by incubation with 300 IU/ml of hyaluronidase and then fixed and stained for both PGRMC1 and DNA using the PGRMC1 antibody (Sigma Chemical Co. Cat No. HPA002877) and 4′,6-diamidino-2-phenylindole (DAPI), respectively. To observe zygotes and preimplantation stage embryos, female mice are primed with eCG and hCG, as described above, and then placed with an adult male after the hCG injection. Twenty-four hours after hCG injection, a vaginal smear is taken from each female mouse and examined for the presence of sperm. Groups of mice that mated are autopsied 24, 48 and 96 hours after hCG injection and the oviducts and uteruses of the autopsied animals are flushed in order to collect zygotes, cleavage-stage embryos and blastocysts, respectively. These embryos are fixed and stained to assess the localization of PGRMC1.

To examine changes in PGRMC1 localization during fertilization and preimplantation development, adult female mice are treated with eCG and hCG. Sixteen hours after hCG, oocytes are collected from the oviducts and incubated with approximately 1 million sperm, as described, for example, in Summers et al., “Mouse embryo development following IVF in media containing either L-glutamine or glycyl-L-glutamine,” Human Reproduction, 20:1364-1371, 2005. The sperm is collected from CD1 male mice as described in Summers et al., “IVF of mouse ova in a simplex optimized medium supplemented with amino acids,” Human Reproduction, 15:1791-1801, 2000. The cultures are examined after 24, 96, and 120 hours after exposure to sperm. The percentage of cleaved embryos (namely, 2-cell embryos after 24 hours) and blastocysts, respectively are determined. After 120 hours after exposure to sperm, the blastocysts are fixed and differentially stained to determine the number of cells within the inner cell mass and trophectoderm, using a protocol described, for example, in Kochhar et al., “In vitro production of cattle-water buffalo (Bos taurus-Bubalus bubalis) hybrid embryos,” Zygote, 10:155-162, 2002. Cell-imaging studies monitoring changes are conducted to evaluate colocalization of PGRMC1 at certain stages of oocyte maturation, fertilization and early preimplantation development with one or more cell structures known to be functionally significant in this process.

### Example 6

### PGRMC1-GFP Expression Vector

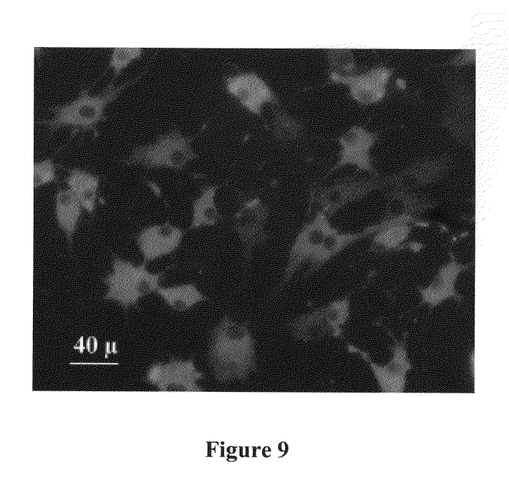

An adenovirus-PGRMC1-GFP expression vector was prepared by isolating total RNA from GL5 cells, a human granulosa cell line, to generate cDNA. The PGRMC1 open reading frame was amplified by PCR. The primers, described in Mansouri et al., “Alterations in the expression, structure and function of progesterone receptor membrane component-1 (PGRMC1) in premature ovarian failure,” Hum. Mol. Gen. 17:3776-3783, 2008, contained an XhoI and a HindIII sites at the ends to facilitate, cloning into the pShuttle-CMV vector. Co-transfection of the linearized pShuttle-CMV-PGRMC1 and pAdTrack DNA was then performed. Viral stocks were amplified, titered and stored at −80° C. As shown in FIG. 9, infection with this adenoviral construct at a MOI of 1×10⁻⁷ was very effective, resulting in nearly 100% of the human granulosa/luteal cells expressing PGRMC1-GFP fusion. Treatment with the adenoviral-PGRMC1-GFP expression vector increased protein levels of PGRMC1-GFP by several fold compared to endogenous PGRMC1 levels. Once transfected, the PGRMC1-GFP continued to be expressed for at least 72 hours post-infection.

### Example 7

### A Method to Determine Functional Significance of PGRMC1 Mutations

Physiological importance of PGRMC1 mutations, including, but not limited to the known mutations H165R PGRMC1 or D120G PGRMC1, is assessed by making PGRMC1 fusion proteins (“fusion proteins”) and injecting them into germinal vesicle stage oocytes or metaphase II oocytes. Alternatively these fusion proteins are transfected into any suitable target cell. In one example, fusion proteins are fusions of wild-type or mutant PGRMC1 sequences with a green fluorescent protein (GFP). If GFP fusion proteins are tested, then GFP is injected as a negative control. It is understood that negative controls are selected based on the type of a fusion protein. Cell-imaging studies according to procedures known to those of ordinary skill in the art are used to monitor localization of the wild-type and mutant fusion proteins and the ability of the injected oocytes to undergo in vitro maturation, fertilization and embryonic development, as discussed in more detail below.

Expression vectors for the wild type and mutant fusion proteins (“expression vectors”) were or are prepared using conventional molecular biology techniques, for example, as described in Peluso I, and Example 6 of this document. The fusion proteins and the negative control, at a concentration of approximately 10 pg in 10 pl, are injected into germinal vesicle stage oocytes as describe in Gordo et al., “Injection of sperm cytosolic factor into mouse metaphase II oocytes induces different developmental fates according to the frequency of [Ca(2+)](i) oscillations and oocyte age,” Biol. Reprod., 62:1370-1379, 2000. The injections result in final intracellular concentrations of the injected material of 0.3 pg/oocyte. After the injections, the oocytes are cultured for 24 hours. The Hoechst 33342 dye is used to stain DNA in living oocytes according to known procedures, described, for example, in Cao, thereby maximizing the GFP fluorescence and still allowing for the determination of the stage of maturation. To determine if the PGRMC1 fusion protein interacts with the DNA, the living oocytes are observed under confocal optics and images are obtained of the fusion protein merged with the DNA.

In order to determine appropriate experimental conditions, injections of various dosages of the wild-type fusion protein are tested and the effectiveness is observed Effectiveness of the injection is determined by monitoring two endpoints. First, the effect of the purified PGRMC1 fusion protein on oocyte maturation, fertilization and embryonic development is monitored. Second, the ability of the wild-type fusion protein to localize to the kinetochore/centromere complex is assessed. If wild-type fusion protein localizes to the kinetochore/centromere complex, thereby mimicking endogenous PGRMC1, then the injection is considered functional. Thus identified experimental conditions are used to assess the effect of the mutated PGRMC1 fusion protein.

If injections of wild-type PGRMC1 fusion protein do not appear to mimic endogenous PGRMC1, troubleshooting of potential experimental problems is performed. For example, in order to overcome a potential problem that the function of the injected fusion protein is adversely affected because it is not endogenously synthesized within the oocytes, injection of expression vectors into oocytes can be employed. For this application, the expression vectors are modified by extending the polyA tail to facilitate protein synthesis in germinal-vesicle stage oocytes, as described, for example, in Aida et. al., “Expression of a green fluorescent protein variant in mouse oocytes by injection of RNA with an added long poly(A) tail,” Mol. Hum. Reprod., 7(11):1039-1046, 2001. Oocyte maturation can be delayed, as needed, by the addition of cAMP analogs, in order to ensure sufficient time to achieve adequate levels of protein expression.

In another example of a potential experimental problem, wild-type PGRMC1 fusion protein fails to localize to the chromosomes, as has been shown for endogenous PGRMC1, due to the presence of the GFP-tag at the C-terminus. It is recognized, for example, that GFP's presence at the C-terminus might interfere with PGRMC1 's interaction with chromosomes. PGRMC1 expression vectors and fusion proteins are therefore generated, in which the GFP-tag is placed on the N-terminus. It is also recognized that a relatively large size (approximately 26 kDa) of the GFP tag can influence its ability of the fusion proteins to localize to the chromosomes. A smaller tag, such as the amino acid sequence YPYDVPDYA, which is known as an HA, can therefore be used to tag the exogenous PGRMC1, and the oocytes are fixed and co-stained with an antibody to HA and DAPI.

Another example of a potential experimental problem to be addressed is depletion of endogenous PGRMC1 levels in order to observe the effects of the experimentally introduced fusion proteins. The depletion is achieved by PGRMC1 siRNA treatment, based on the experimental approaches discussed, for example, in Peluso I, Peluso II and III. An alternative to siRNA treatment is the development of a transgenic mouse in which PGRMC1 is depleted conditionally from the oocyte.

Experimental studies according to the procedures similar to those described above in Examples 4 and 5 are conducted. Cell-imaging studies monitoring changes are conducted to evaluate colocalization of mutant PGRMC1 at certain stages of oocyte maturation, fertilization and early preimplantation development with one or more cell structures known to be functionally significant in this process in order to assess functional significance of PGRMC1 mutations.

### Example 8

### Selection of Patients for Studies in Human Subjects

Human subjects were selected from the patients of a clinic having a large number of patients undergoing in vitro fertilization protocols, a high success rate in terms of number of births per embryo transfer and a computerized database in which patient information can be retrieved. For example, the analysis of one suitable clinic's patient records revealed that 48% of all embryo transfers in the clinic resulted in a live birth, therefore indicating a high level of patient management or laboratory errors.

To minimize variation among the human subjects, only women 37 years of age or less with a diagnosis of either tubal factor only or unexplained infertility were enrolled in certain studies (Group I). In other studies, the human subjects were further limited to the female patients who responded normally to gonadotropin treatment but failed to conceive on their first in vitro fertilization attempt and were undergoing a second in vitro fertilization protocol (Group II). A normal response to gonadotropin treatment was generally characterized by the presence of peak estradiol levels of >700 pg/ml, 2 or more follicles greater than 18 mm in diameter and over 4 oocytes retrieved. A detailed analysis of the patients' records at the clinic discussed above revealed that, for a selected year, 224 patients with unexplained or tubal infertility failed to conceive, but had a normal response to gonadotropin. Of these patients, 194 (87%) underwent a second in vitro fertilization cycle. Women enrolled in an egg donor program who were 37 years of age or less and were responsive to gonadotropin treatment were used as controls, because these women were selected so that they have fertilization rates ≧80%.

### Example 9

### Characterization of Patients for Studies in Human Subjects

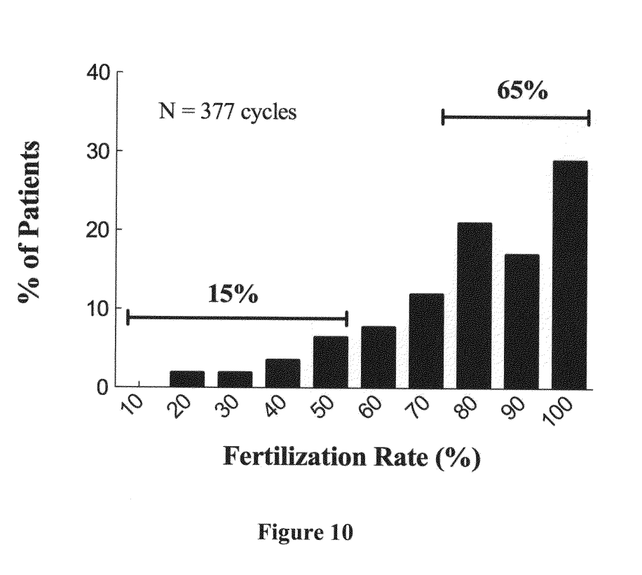

Group II human patient subjects were classified, based on the data from these patients' first in vitro fertilization cycle, as those with low (≦50%) or high (≧80%) fertilization rates. A distribution analysis revealed that fertilization rates were not normally distributed but skewed in Group II, as shown in FIG. 10. About 15% of Group II subjects had oocytes with low fertilization potential, and 65% of Group II subjects had oocytes with high fertilization potential.

### Example 10

### Studies of PGRMC1 in Human Subjects

Studies in human subjects are conducted to test levels of expression and/or genetic structure of PGRMC1 in female patients undergoing fertility treatments, such as in vitro fertilization, and to correlate changes in PGRMC1 expression and genetic structure to the ability of the patients' oocytes to undergo fertilization and embryonic development in vitro. A population of infertility patients is identified, in which oocytes fail to either fertilize and/or undergo embryonic development during in vitro fertilization procedures. These patients are classified as low fertility patients. Molecular and cell biological techniques are used to monitor the level of expression and genetic structure of PGRMC1 in these selected patients undergoing infertility treatment.

### Example 11

### Studies of PGRMC1 from Granulosa Cells of Human Subjects

Human subjects selected as discussed, for example, in Example 8, undergo, as a part of their in vitro fertilization treatment, an ovulation induction protocol, which consists of treatment with the Gonadotropin Release Hormone analog, Lupron during the luteal phase to suppress ovarian function. Once ovarian function is suppressed, the subjects are treated with recombinant Follicle Stimulating Hormone until 2 or more follicles ≧18 mm in diameter are observed. Human chorionic gonadotropin is then administered and the oocytes and granulosa/luteal cells are retrieved 36 h later. As a part of their in vitro fertilization treatment, follicular aspirates are obtained from each patient. Granulosa/luteal cells are obtained from the aspirates and centrifuged at 250×g for 10 min. The supernatant is discarded and the cell pellet is resuspended in phosphate-buffered saline (PBS).

The cells are layered on a Histopaque-1077 gradient and centrifuged at 300×g for 40 min. The cell layer is removed and washed twice in serum-free medium and then used to monitor granulosa cells PGRMC1 levels and genetic structure, steroidogenic capacity and viability. Total RNA is isolated from an aliquot of cells of each patient. PGRMC1 will be assessed by two different procedures. First, some of the isolated RNA from each sample is used to determine the amount of PGRMC1 mRNA by real time PCR using a protocol similar to a published protocol, described, for example, in Peluso III. PGRMC1 mRNA levels are normalized against actin as an internal control. mRNA levels indicate the amount of PGRMC1 that is expressed in the granulosa cells of the subjects. PGRMC1 mRNA levels from the egg donors are used as a normal control and are averaged, a standard deviation is calculated and a lower 95% confidence limit is determined. Any infertility patient whose PGRMC1 mRNA level is outside of the 95% confidence limit is considered to have abnormally PGRMC1.

DNA and RNA are also used in a RT-PCR protocol that amplify the entire coding sequence of PGRMC1 using primer pairs previously described, for example, in Peluso III. The PCR product is sequenced, providing information on the genetic structure of PGRMC1 from the granulose cells of the subjects.

The results described in this section show that alterations in either the level or genetic structure of PGRMC1 are associated with poor fertility in human subjects.

### Example 12

### Studies of PGRMC1 from Lymphocytes of Human Subjects

For all patients enrolled in this study, lymphocytes are isolated using Ficoll-Paque PREMIUM gradient centrifugation per manufacturer's instruction (GE Healthcare, Inc.) and DNA, RNA and protein isolated using the TrioMol isolation kit from GenScript (Piscataway, N.J.). This kit can be used to isolate DNA, RNA and protein from the same blood sample and the isolated DNA/RNA and protein are suitable for PCR and Western blot protocols, respectively. To ensure that any changes in the level or structure of PGRMC1 observed in lymphocytes are also present within the ovary, mRNA is isolated from granulosa cells harvested from each patient at the time of oocyte retrieval per standard protocol, described, for example, in Engmann et al., “Progesterone regulation of human granulose/luteal cell viability by an RU486-independent mechanism,” J. Clin. Endocrinol. Metab. 91(12):4962-4968, 2006. DNA, RNA and protein isolated from both lymphocytes and granulosa cells are analyzed as outlined below.

Analysis of PGRMC1 mRNA, genetic structure and protein levels is conducted. PGRMC1 is assessed by at least three different procedures. First, some of the isolated RNA from each sample is used to determine the amount of mRNA by real time PCR using a known protocol, such as the one described in Peluso III. PGRMC1 mRNA levels are normalized against actin as a control. Thus obtained experimental data on the mRNA amounts provides information on the amount of expressed PGRMC1. DNA is used in a PCR protocol amplifies the entire coding sequence of PGRMC1 (i.e. Exons 1-3) using primer pairs as previously described, for example, in Mansouri. The PCR products are sequenced, which provides information on the genetic structure of PGRMC1.

The protein isolated from each sample is processed for Western blot analysis using known procedures. PGRMC1 antibody, such as a rabbit antibody commercially available from Sigma Chemical Co., and anti-rabbit IR Dye 800 (Li-Cor Bioscience, Lincoln, Nebr.) are used as the primary and secondary antibodies, respectively. The blot is simultaneously probed with a GAPDH mouse primary antibody and an anti-mouse IRDye 700 secondary antibody. The Western blots are imaged in a quantitative manner using the Odyssey Infrared imaging system from Li-Cor Bioscience (Lincoln, Nebr.). GAPDH serves as a loading control. This analysis provides information on an amount of PGRMC1 expressed and on the changes in its molecular weight. If changes in the molecular weight are observed, these post-translational modifications such as phosphorylations, are assessed according to known experimental procedures.

Other patient populations are also analyzed. The experimental data are statistically analyzed. Functional significance of those genetic alterations of PGRMC1 that are not suitable for statistical analysis because they are not observed at a high enough frequency is assessed as outlined in Examples 7 of this document.

The experimental results obtained from at least some of the studies described above indicate that certain genetic changes in PGRMC1 alter a woman's fertility, for example, a woman's ability to generate fertilizable oocytes. Mutations in PGRMC1 affecting a woman's fertility or an ability to generate fertilizable oocytes are identified.

### Example 13

### Clinical Studies in Human Subjects

Clinical studies in human subjects are conducted to correlate PGRMC1 expression, structure and sequence with in vitro fertilization outcomes and fertility in general. One or more studies enroll a relatively large number of subjects that is sufficient to consider the influence of different racial backgrounds and other potential factors. The results obtained from at least some of the clinical studies used to plan the most effect treatment for other women undergoing in vitro fertilization protocols.

### Example 14

### A Test to Assess Fertility in Women

A test is developed that determines, based on a sample obtained from a patient, whether the patient's PGRMC1 contains variations that correlate with the in vitro fertilization outcomes and fertility in general. The results obtained from such a test may be used to calculate the values evaluating fertility in women and predicting the outcome of fertility treatments, such as, but not limited to, in vitro fertilization procedures.

### Example 15

### Elevated PGRMC1 mRNA Levels Correlate with Low Oocyte Numbers in Female Infertility Patients

A study conducted on a population of infertility patients showed that elevated PGRMC1 mRNA levels in a patient correlated with low oocyte numbers generated by the patient.

While this invention has been described in detail with regard to embodiments thereof, it should be understood that variations and modifications can be made without departing from the spirit and scope of the invention described herein and/or defined in the following claims.

All the documents cited herein are incorporated by reference in their entirety.

What is claimed is:

1. A method of evaluating a level of fertility of a human comprising:
obtaining a sample from the human;
determining a PGRMC1 characteristic in the sample; and,
comparing the determined PGRMC1 characteristic to a baseline PGRMC1 characteristic,
wherein a variation between the determined PGRMC1 characteristic and the baseline characteristic indicates the level of fertility of the human.

2. The method of claim 1, wherein the PGRMC1 characteristic is one or more of PGRMC1 expression, transcription, translation, amino acid sequence, nucleic acid sequence, post-translational modification, cell localization or tissue localization.

3. The method of claim 1, wherein the sample is a cell sample or a tissue sample.

4. The method of claim 1, wherein the sample is a blood sample, a lymphocyte sample, an ovarian tissue sample, an oocyte sample, a testicular tissue sample or a sperm sample.

5. The method of claim 1, wherein the human is a female.

6. The method of claim 1, wherein the human is a male.

7. The method of claim 1, wherein the PGRMC1 characteristic is a level of PGRMC1 expression and wherein the variation is an altered expression that indicates reduced fertility of the human.

8. The method of claim 1, wherein the PGRMC1 characteristic is a nucleic acid sequence and the variation is a variation of the nucleic acid sequence that indicates reduced fertility of the human.

9. The method of claim 8, wherein the variation is at least one of H165R mutation or D120G mutation in the nucleic acid sequence of PGRMC1.

10. The method of claim 1, wherein the level of fertility is a capacity of the human to produce functional gametes.

11. A method of evaluating a capacity of a human to produce functional gametes, comprising determining a characteristic of PGRMC1 of a human, wherein the characteristic indicates the capacity of the human to produce the functional gametes.

12. The method of claim 11, wherein the PGRMC1 characteristic is one or more of a level of expression, a level of transcription, a level of translation, amino acid sequence, nucleic acid sequence, post-translational modification, cell localization or tissue localization.

13. The method of claim 11, wherein the human is a female and the functional gametes are fertilizable oocytes.

14. The method of claim 11, wherein the PGRMC1 characteristic is a level of PGRMC1 expression.

15. The method of claim 11, wherein the PGRMC1 characteristic is a nucleic acid sequence.

16. A method of evaluating a probability of an outcome of a fertility treatment in a human patient, comprising determining a characteristic of PGRMC1 of the human patient, wherein the characteristic indicates the probability of the outcome of the fertility treatment in the human patient.

17. The method of claim 16, wherein the PGRMC1 characteristic is one or more of a level of expression, a level of transcription, a level of translation, amino acid sequence, nucleic acid sequence, post-translational modification, cell localization or tissue localization.

18. The method of claim 16, wherein the PGRMC1 characteristic is a level of PGRMC1 expression.

19. The method of claim 16, wherein the PGRMC1 characteristic is a nucleic acid sequence.

20. The method of claim 16, wherein the PGRMC1 characteristic is at least one of H165R mutation or D120G mutation in the nucleic acid sequence of PGRMC1.
